(19) United States
(12) Patent Application Publication
(10) Pub. No.: US 2025/0232841 A1
(43) Pub. Date: Jul. 17, 2025

(54) DESIGNING PROTEINS BY JOINTLY MODELING SEQUENCE AND STRUCTURE

(71) Applicant: DeepMind Technologies Limited, London (GB)

(72) Inventors: Simon Kohl, London (GB); John Jumper, London (GB); Andrew W. Senior, London (GB); Vinicius Zambaldi, London (GB); Rosalia Galiazzi Schneider, London (GB); Russell James Bates, London (GB); Gabriella Hayley Stanton, London (GB); Robert David Fergus, New York, NY (US); David La, London (GB); David William Saxton, London (GB); Fabian Bernd Fuchs, , United Kingdom (GB)

(21) Appl. No.: 18/703,737

(22) PCT Filed: Nov. 21, 2022
(86) PCT No.: PCT/EP2022/082678

§ 371 Date: Apr. 23, 2024

Related U.S. Application Data
(60) Provisional application No. 63/282,526, filed on Nov. 23, 2021.

(51) Int. Cl. G16B 40/00; G06N 3/045; G16B 15/20

(52) U.S. Cl. CPC G16B 40/00; G06N 3/045; G16B 15/20

(57) ABSTRACT

Methods, systems, and apparatus, including computer programs encoded on a computer storage medium, for designing a protein by jointly generating an amino acid sequence and a structure of the protein. In one aspect, a method comprises: generating data defining the amino acid sequence and the structure of the protein using a protein design neural network, comprising, for a plurality of positions in the amino acid sequence: receiving the current representation of the protein as of the current position: processing the current representation of the protein using the protein design neural network to generate design data for the current position that comprises: (i) data identifying an amino acid at the current position, and (ii) a set of structure parameters for the current position; and updating the current representation of the protein using the design data for the current position.

### BACKGROUND

This specification relates to designing proteins.

A protein includes a sequence of amino acids. An amino acid is an organic compound which includes an amino functional group and a carboxyl functional group, as well as a side-chain (i.e., group of atoms) that is specific to the amino acid.

Protein folding refers to a physical process by which a sequence of amino acids folds into a three-dimensional configuration. The structure of a protein defines the three-dimensional (3D) configuration of the atoms in the amino acid sequence of the protein after the protein undergoes protein folding. When in a sequence linked by peptide bonds, the amino acids may be referred to as amino acid residues.

Predictions can be made using machine learning models. Machine learning models receive an input and generate an output, e.g., a predicted output, based on the received input. Some machine learning models are parametric models and generate the output based on the received input and on values of the parameters of the model. Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes a protein design system implemented as computer programs on one or more computers in one or more locations for designing a protein by jointly generating an amino acid sequence and a structure of the protein using an autoregressive protein design neural network. A design for a protein designed by the system may be expressed as the amino acid sequence of the protein, the structure of the protein, or both.

Throughout this specification, an “embedding” can refer to an ordered collection of numerical values, e.g., a vector, matrix, or other tensor of numerical values.

A “data element” can refer to, e.g., a token, a numerical value, or an embedding.

A “block,” in a neural network, can refer to a portion of the neural network, e.g., a sub-network of the neural network that includes one or more neural network layers.

According to a first aspect, there is provided a method performed by one or more computers for designing a protein by jointly generating an amino acid sequence and a structure of the protein, the method comprising: initializing a current representation of the protein; generating data defining the amino acid sequence and the structure of the protein using a protein design neural network, comprising, for a plurality of positions in the amino acid sequence: receiving the current representation of the protein as of the current position; processing the current representation of the protein using the protein design neural network to generate design data for the current position that comprises: (i) data identifying an amino acid at the current position, and (ii) a set of structure parameters for the current position that characterize a three-dimensional spatial configuration of the amino acid at the current position; updating the current representation of the protein using the design data for the current position; and providing the current representation of the protein for use in generating design data for a next position in the amino acid sequence of the protein; and outputting the data defining the amino acid sequence and the structure of the protein.

In some implementations, for each current position after a first position in the amino acid sequence: the current representation of the protein as of the current position comprises a representation of respective design data for each preceding position that precedes the current position in the amino acid sequence; and the design data for each preceding position comprises: (i) data identifying an amino acid at the preceding position, and (ii) a set of structure parameters for the preceding position.

In some implementations, for each current position in the amino acid sequence, the current representation of the protein as of the current position further comprises conditioning data that specifies desired characteristics of the protein.

In some implementations, the protein design neural network comprises an encoder neural network, an amino acid neural network, and a structure neural network; and wherein processing the current representation of the protein using the protein design neural network to generate design data for the current position comprises: processing the current representation of the protein using the encoder neural network to generate an encoded representation of the protein; and processing an input comprising the encoded representation of the protein using the amino acid neural network to generate the data identifying the amino acid at the current position; and processing an input comprising the encoded representation of the protein using the structure neural network to generate the structure parameters for the current position.

In some implementations, processing an input comprising the encoded representation of the protein using the amino acid neural network to generate data identifying the amino acid at the current position comprises: processing the input comprising the encoded representation of the protein using the amino acid neural network to generate a probability distribution over a set of amino acids; and selecting the amino acid at the current position using the probability distribution over the set of amino acids.

In some implementations, selecting the amino acid at the current position using the probability distribution over the set of amino acids comprises: sampling the amino acid at the current position in accordance with the probability distribution over the set of amino acids.

In some implementations, processing the input comprising the encoded representation of the protein using the structure neural network to generate the structure parameters for the current position comprises: processing the input comprising the encoded representation of the protein using the structure neural network to generate a probability distribution over a set of structure parameters; and selecting the structure parameters for the current position using the probability distribution over the set of structure parameters.

In some implementations, selecting the structure parameters for the current position using the probability distribution over the set of structure parameters comprises: sampling the structure parameters for the current position in accordance with the probability distribution over the set of structure parameters.

In some implementations, the structure neural network processes an input that comprises both: (i) the encoded representation of the protein, and (ii) data identifying the amino acid at the current position.

In some implementations, processing the current representation of the protein using the encoder neural network to generate the encoded representation of the protein comprises: processing the current representation of the protein to generate a collection of embeddings that includes a respective embedding representing an amino acid at each preceding position that precedes the current position in the amino acid sequence; updating the collection of embeddings using one or more self-attention operations; and after updating the collection of embeddings using the one or more self-attention operations, generating the encoded representation of the protein based on the collection of embeddings.

In some implementations, the self-attention operations are conditioned on respective structure parameters for each preceding position that precedes the current position in the amino acid sequence.

In some implementations, updating the current representation of the protein using the design data for the current position comprises: updating the current representation of the protein to include a representation of the design data for the current position.

In some implementations, for each of the plurality of positions in the amino acid sequence, the structure parameters for the position comprise backbone torsion angles.

In some implementations, initializing the current representation of the protein comprises: initializing the current representation of the protein to include conditioning data that specifies desired characteristics of the protein.

In some implementations, the conditioning data specifies desired characteristics of the amino acid sequence of the protein, the structure of the protein, or a biological function of the protein.

In some implementations, the conditioning data defines a protein fragment to be extended by the protein generated using the protein design neural network.

In some implementations, the conditioning data defines a target protein that provides a binding target for the protein generated using the protein design neural network.

In some implementations, the method further comprises providing the protein generated using the protein neural network to be physically synthesized.

According to another aspect, there is provided a system comprising: one or more computers; and one or more storage devices communicatively coupled to the one or more computers, wherein the one or more storage devices store instructions that, when executed by the one or more computers, cause the one or more computers to perform operations of methods described herein.

According to another aspect, there are provided one or more non-transitory computer storage media storing instructions that when executed by one or more computers cause the one or more computers to perform operations of the methods described herein.

According to another aspect, there is provided a method for producing a protein, comprising: generating data defining an amino acid sequence and a structure of a protein using the methods described herein; and synthesizing a protein having the amino acid sequence.

Particular embodiments of the subject matter described in this specification can be implemented so as to realize one or more of the following advantages.

The protein design system described in this specification can use a protein design neural network to jointly generate an amino acid sequence and a structure of a protein, e.g., to achieve a set of design criteria specified by conditioning data provided by a user. That is, the protein design neural network jointly models the amino acid sequence and the structure of the protein, rather than, e.g., modeling the amino acid sequence in isolation from the protein structure. Jointly modeling the amino acid sequence and the structure of a protein enables the protein design neural network to generate richer internal representations, e.g., at the hidden layers of the protein design neural network, that implicitly encode both sequence and structural features of the protein. The richer implicit reasoning enabled by jointly modeling sequence and structure can allow the protein design neural network to be trained to achieve an acceptable level of performance (e.g., in generating stable proteins that achieve specified design criteria) more rapidly (e.g., over fewer training iterations) and using less training data. The protein design neural network thus enables reduced use of computational resources, e.g., memory and computing power, during training.

The protein design neural network autoregressively generates data defining the amino acid sequence and the structure of a protein. More specifically, the protein design neural network sequentially generates the amino acid and the structure parameters for each position in the amino acid sequence of a protein, starting from a first position, and in accordance with the ordering of the positions. To generate the amino acid and the structure parameters for a current position, the protein design neural network processes an input that defines the amino acids and the structure parameters previously generated for any preceding positions in the amino acid sequence of the protein. Thus the protein design neural network incrementally constructs the amino acid sequence and the structure of a protein, and at each position in the amino acid sequence, accounts for the context provided by the amino acids and the structure parameters generated for any preceding positions. The autoregressive architecture of the protein design neural network can enable the protein neural network to generate proteins that are more realistic (e.g., more stable) and that better satisfy design criteria.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example protein design system.

FIG. 2 illustrates examples of operations performed by a protein design system to generate design data for a current position in an amino acid sequence of a protein.

FIG. 3 shows an example architecture of a protein design neural network.

FIG. 4 shows an example architecture of an encoder neural network that is included in a protein design neural network.

FIG. 5 is an illustration of an unfolded protein and a folded protein.

FIG. 6 illustrates backbone torsion angles and side chain torsion angles in an amino acid.

FIG. 7 is a flow diagram of an example process for designing a protein by jointly generating an amino acid sequence and a structure of the protein.

Like reference numbers and designations in the various drawings indicate like elements.

### DETAILED DESCRIPTION

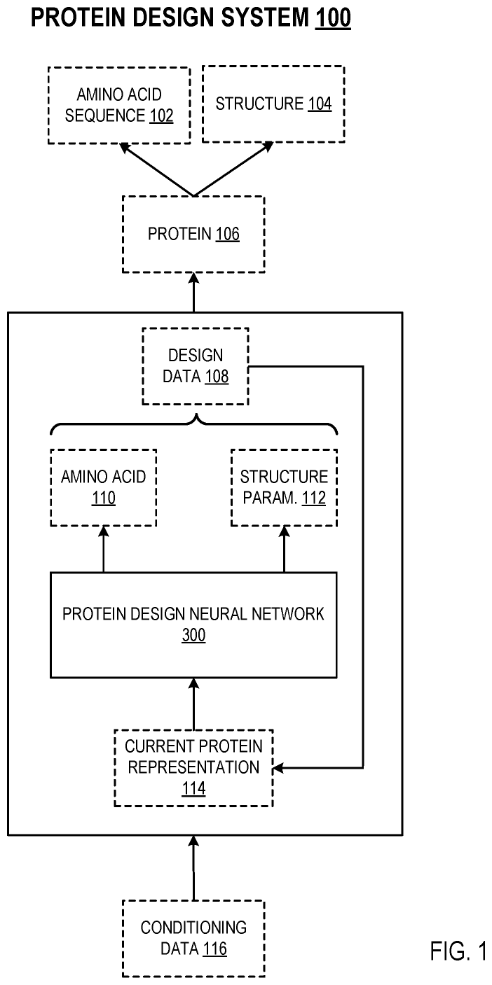

FIG. 1 shows an example protein design system 100. The protein design system 100 is an example of a system implemented as computer programs on one or more computers in one or more locations in which the systems, components, and techniques described below are implemented.

The protein design system 100 is configured (and trained) to generate data defining a protein 106, in particular: (i) an amino acid sequence 102 of the protein 106, and (ii) a structure 104 of the protein 106. Optionally, the protein design system 100 can receive conditioning data 116 that specifies desired characteristics of the protein to be generated, and the protein design system 100 can condition the generation of the protein 106 on the conditioning data 116, as will be described in more detail below.

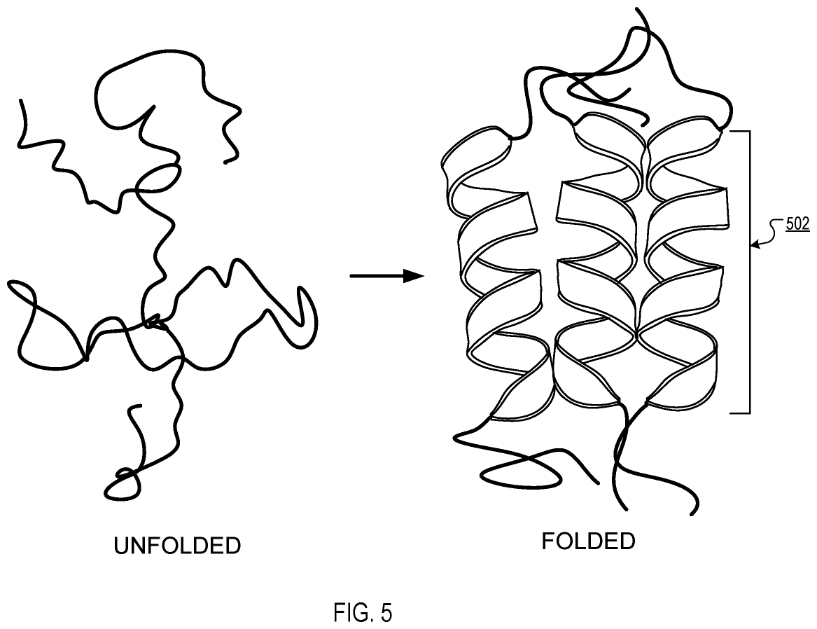

The protein structure 104 refers to the three-dimensional configuration of the protein 106 after the protein 106 undergoes protein folding. (FIG. 5 provides an illustration of an unfolded protein and a folded protein). The protein structure 104 is defined by a respective set of one or more structure parameters for each position in the amino acid sequence 102 of the protein 106. The respective structure parameters for each position in the amino acid sequence of the protein collectively define the structure of the protein.

The structure parameters for a position in the amino acid sequence 102 characterize the 3D spatial configuration of the amino acid at the position in the structure of the protein. The structure parameters for a position in the amino acid sequence can be represented in a variety of possible ways. A few example representations of the structure parameters for a position in the amino acid sequence 102 are described next.

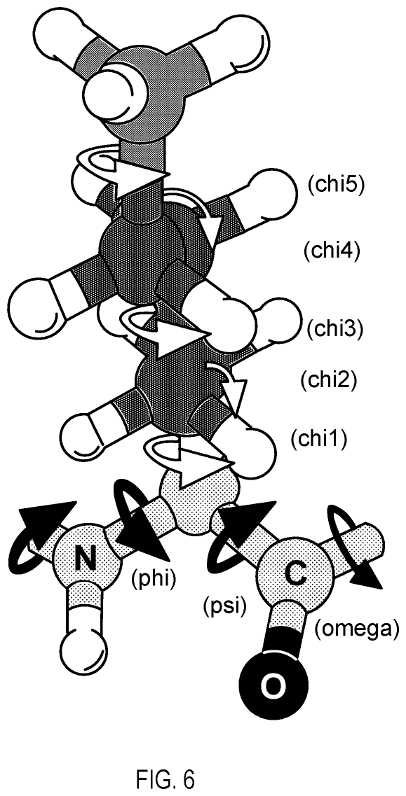

In one example, the structure parameters for a position in the amino acid sequence can include a set of backbone torsion (dihedral) angles of the bonds connecting the backbone atoms in the amino acid at the position. (The backbone atoms in an amino acid can refer to the linked series of nitrogen, alpha carbon, and beta carbon atoms in the amino acid). For example, the torsion angles can include a psi-angle (e.g., of the alpha carbon-beta carbon bond), an omega-angle (e.g., of the beta carbon-nitrogen bond), and a phi-angle (e.g., of the nitrogen-alpha carbon bond). In general a protein can be characterized by the two torsion angles, psi and phi; the omega angle is optional as it is typically constrained e.g. to 180° (or) 0°. FIG. 6 provides an illustration of the backbone torsion angles of an amino acid (the chi-angle is described later).

In another example, the structure parameters for a position in the amino acid sequence can include location parameters that define a 3D spatial location of a specified atom in the amino acid at the position. The specified atom can be the alpha carbon atom in the amino acid. The location parameters for an amino acid can be represented in any appropriate coordinate system, e.g., a three-dimensional [x, y, z] Cartesian coordinate system.

In another example, the structure parameters for a position in the amino acid sequence can include rotation parameters. The rotation parameters can specify the predicted “orientation” of the amino acid in the structure of the protein. More specifically, the rotation parameters can specify a 3D spatial rotation operation that, if applied to the coordinate system of the location parameters, causes the three backbone atoms in the amino acid to assume fixed positions relative to the rotated coordinate system. The rotation parameters for an amino acid can be represented, e.g., as an orthonormal 3× 3 matrix with determinant equal to 1.

Generally, the location and rotation parameters for a position in the amino acid sequence jointly define an egocentric reference frame for the amino acid at the position. In this reference frame, the side chain for each amino acid may start at the origin, and the first bond along the side chain (i.e., the alpha carbon-beta carbon bond) may be along a defined direction.

In another example, the structure parameters for a position in the amino acid sequence can include side chain parameters defining the 3D spatial positions of the atoms in the side chain of the amino acid at the position. For example, the structure parameters can include side chain torsion angles of the bonds connecting the side chain atoms in the side chain, e.g., a chi1-angle, chi2-angle, chi3-angle, and chi4-angle. FIG. 6 provides an illustration of the side chain torsion angles of an amino acid.

Generally, the protein design system 100 generates a protein 106 in response to receiving a “query,” i.e., a request (e.g., from a user) to generate a protein 106. Optionally, the query may include conditioning data 116 specifying (desired) characteristics of the protein to be generated. If no conditioning data is included the system can be used to generate protein designs for proteins that are similar to, e.g. drawn from the same distribution as, those used to train the system. Thus the system may be used to design proteins with a characteristic or attribute of any particular type, e.g. as described below with reference to the conditioning data, by training the system using (mainly or only) proteins with this characteristic or attribute.

A user can query the protein design system 100, e.g., by way of a user interface or an application programming interface (API) made available by the protein design system 100. The user may be remotely located from the protein design system. For example, a remotely located user can transmit a query to the protein design system 100 by way of a data communication network, and the protein design system 100 can transmit data defining a protein generated in response to the query to the user over the data communications network. The data communication network may be, e.g., a local area network (LAN), a wide area network (WAN), or the internet.

The protein design system 100 can be queried any appropriate number of times, e.g., one time, 100 times, or 1000 times, and generate a respective protein in response to each query. The protein design system 100 can generate a protein 106 using operations that involve probabilistic sampling (as will be described in more detail below), and therefore the protein design system 100 may generate a different protein in response to each query, even if each query includes the same conditioning data.

The conditioning data 116 can specify any of a variety of possible characteristics of the protein to be generated by the protein design system 100. A few examples of conditioning data 116 are described next. The system is not limited to any particular format or content of the conditioning data. For example the system can be trained based on conditioning data with a particular format or content, and the same format or content of conditioning data can be used in inference.

In one example, the conditioning data 116 can define one or more attributes of the amino acid sequence of the protein to be generated by the protein design system 100. For example, the conditioning data 116 can characterize a length of the amino acid sequence, i.e., a number of amino acids in the amino acid sequence. In a particular example, the conditioning data 116 can specify a range of lengths (e.g., 70-80 amino acids), such that the generated amino acid sequence should have a length within the specified range of lengths. As another example, the conditioning data 116 can characterize a distribution of the types of amino acids in the amino acid sequence, e.g., the conditioning data 116 can specify that certain amino acids (e.g., cysteine) should not occur in the amino acid sequence, or the conditioning data 116 can specify that no more than a specified fraction of the amino acids in the amino acid sequence should be hydrophobic.

In another example, the conditioning data 116 can define one or more attributes of the structure of the protein to be generated by the protein design system 100. For example, the conditioning data 116 can specify a number of times that a particular structural motif, e.g. secondary structure, should occur in the protein structure. Examples of structural motifs can include, e.g., beta sheets and alpha helices. As another example, the conditioning data 116 can specify respective structure parameters for some or all of the positions in the amino acid sequence of the protein to be generated by the protein design system 100. As another example, the conditioning data 116 can specify which positions in the amino acid sequence should be occupied by amino acids positioned at the surface of the protein structure, or which positions in the amino acid sequence should be occupied by amino acids positioned in the core of the protein structure.

In another example, the conditioning data 116 can define a biological function of the protein to be generated by the protein design system, e.g., by specifying a particular biological function from a set of biological functions. The set of biological functions can include, e.g., antibody functions, enzyme functions, messenger functions, structural functions, or transport/storage functions.

In another example, the conditioning data 116 can define the amino acid sequence and the structure of a protein fragment to be extended by the protein design system. That is, in this example, the protein design system 100 performs “scaffolding” by generating an extension of the protein fragment defined by the conditioning data 116.

In another example, the conditioning data 116 can define the sequence and the structure of a “target” protein, such that the protein design system 100 generates a “binder” protein that is predicted to bind to the target protein.

In other examples, the conditioning data 116 can characterize, e.g., a protein family or a host species of the protein to be generated by the protein design neural network.

The protein design system 100 can generate a protein, i.e. a design for a protein, e.g., in response to receiving a request to generate a protein, using a protein design neural network 300. Where conditioning data 116 is used, the protein will in general exhibit the characteristics or attributes specified by the conditioning data.

The protein design neural network 300 can jointly generate both the amino acid sequence 102 and the structure 104 of the protein 106. More specifically, the protein design neural network 300 can generate, for each position in the amino acid sequence 102 starting from the first position and in accordance with the ordering of the positions, data defining: (i) an amino acid 100 at the position, and (ii) structure parameters 112 for the position. That is, the protein design neural network 300 can sequentially generate data defining an amino acid 100 and structure parameters 112 for each position, starting from the first position, in the amino acid sequence 102. For convenience, data defining: (i) the amino acid 100 at a position, and (ii) the structure parameters for the position, may be jointly referred to herein as “design data” 108 for the position.

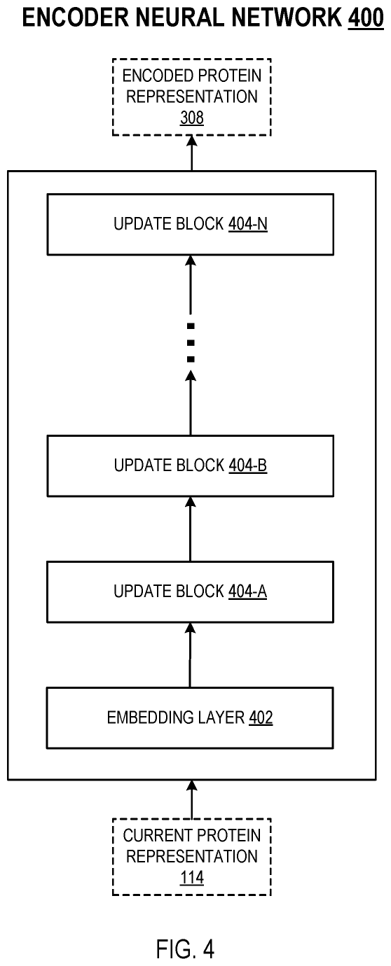

To generate the design data 108 for a current position in the amino acid sequence 102 of the protein 106, the protein design neural network processes a current protein representation 114. Generally, the current protein representation 114 represents at least the respective design data (i.e., amino acids and structure parameters) for any positions preceding the current position in the amino acid sequence 102. The current protein representation 114 can be represented in any appropriate numerical format, e.g., as an ordered collection of numerical values, e.g., as a matrix or other tensor of numerical values. An example format of a current protein representation is described in more detail below with reference to FIG. 4.

The protein design neural network 300 can process the current protein representation 114 to generate data defining: (i) a probability distribution over a set of amino acids, and (ii) a probability distribution over a set of structure parameters. The protein design system 100 can select the amino acid 100 for the current position in the amino acid sequence 102 using the probability distribution over the set of amino acids, e.g., by sampling an amino acid in accordance with the probability distribution. The protein design system 100 can select the structure parameters for the current position in the amino acid sequence 102 using the probability distribution over the set of structure parameters, e.g., by sampling structure parameters in accordance with the probability distribution.

After the protein design neural network 300 generates the design data 108 for the current position, the protein design system 100 updates the current protein representation 114 using the design data 108 for the current position. For example, protein design system 100 can augment the current protein representation 114 to include a representation of the design data 108 for the current position. Then, the protein design system 100 can provide the current protein representation 114, i.e., which has been updated using the design data 108 for the current position, to the protein design neural network 300 to generate design data 108 for the next position in the amino acid sequence 102. Thus the protein design neural network 300 “autoregressively” generates respective design data 108 for each position in the amino acid sequence 102, i.e., by generating the design data 108 for each position based on respective design data generated for any preceding positions in the amino acid sequence 102.

The protein design system 100 can initialize the current protein representation 114, i.e., prior the current protein representation being processed by the protein design neural network 300 to generate the design data for the first position in the amino acid sequence 102, in any appropriate way. For example, the protein design system 100 can initialize the current protein representation 114 to a default representation. As another example, if the protein design system 100 receives conditioning data 116, then the protein design system 100 can initialize the current protein representation 114 to include the conditioning data 116. Example techniques for initializing the current protein representation 114, and for updating the current protein representation using design data for a position in the amino acid sequence 102, are described in more detail below with reference to FIG. 4.

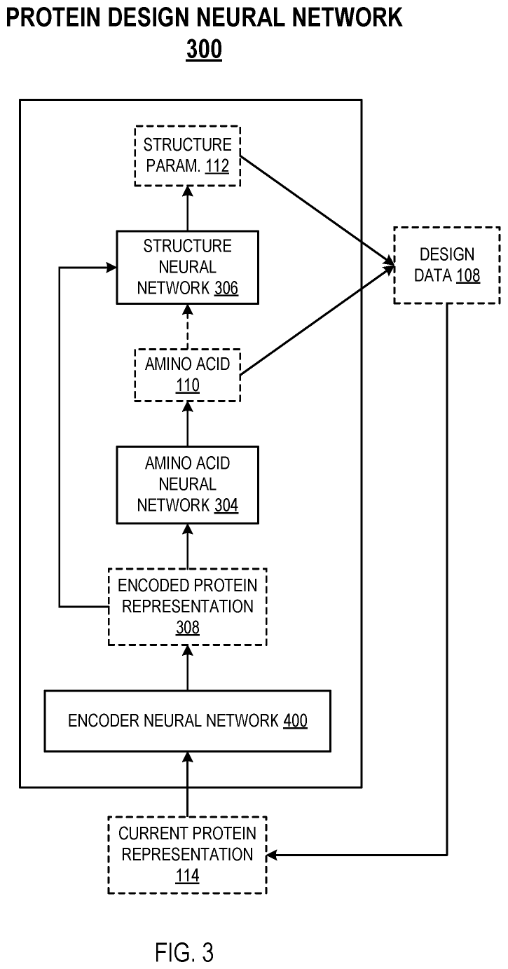

The protein design neural network 300 can have any appropriate neural network architecture that enables it to perform its described functions, e.g., processing a current protein representation 114 to generate design data 108 for a position in an amino acid sequence of a protein. In particular, the protein design neural network can include any appropriate types of neural network layers (e.g., fully-connected layers, convolutional layers, attention layers, etc.) in any appropriate numbers (e.g., 5 layers, 10 layers, or 100 layers) and connected in any appropriate configuration (e.g., as a linear sequence of layers). An example architecture of the protein design neural network 300 is described in more detail below with reference to FIG. 3.

The protein design system 100 can continue generating design data 108 for successive positions in the amino acid sequence 102, using the protein design neural network 300, until a termination criterion is satisfied. The termination criterion can be any appropriate criterion. A few examples of possible termination criteria are described next.

In one example, as part of generating the design data 108 for each position in the amino acid sequence 102, the protein design neural network 300 can generate an additional output that defines whether the current position in the last position in the amino acid sequence 102. In response to the protein design neural network 300 generating an output defining the current position as the last position in the amino acid sequence 102, the protein design system 100 can determine that a termination criterion is satisfied. An example of a protein design neural network that generates an additional output that defines whether the current position is the last position in the amino acid sequence is described in more detail below with reference to FIG. 3.

In another example, the protein design system 100 can determine that a termination criterion has been satisfied once design data 108 has been generated for a predefined number of positions in the amino acid sequence 102. That is, the protein design system 100 can be configured to generate a protein 106 having a predefined length.

After determining that a termination criterion is satisfied, the protein design system 100 can output data defining the amino acid sequence 102 and the structure 104 of the protein 106.

The protein design system 100 can, as described above, generate a binder protein that is predicted to bind to a target protein defined by the conditioning data 116. That is, the protein design system 100 can generate a binder protein that spatially “packs against” the target protein. However, in implementations where the protein design neural network 300 is configured to generate structure parameters 112 represented as backbone torsion angles, the 3D spatial position of each amino acid is defined relative to the 3D spatial position of the preceding amino acid. Moreover, the 3D spatial position of the first amino acid generated by the protein design neural network may be initialized at a default position (e.g., at the origin). The default 3D spatial position of the first amino acid subsequently constrains the 3D spatial positions of the subsequent amino acids, and thus the protein design neural network 300 may find it difficult to generate a binder protein that is appropriately spatially positioned to pack against the target protein.

To address this issue, the protein design system 100 can designate a predefined number of the first positions in the amino acid sequence generated by the protein design neural network 300 as being “linker” positions, while the subsequent positions are designated as being “binder” positions. The protein design system 100 can define each linker position as being occupied by a predefined “linker” amino acid, e.g., glycine, that is known to achieve a highly flexible 3D structure. That is, the protein design system 100 can define the amino acid output of the protein design neural network for the linker positions to be the predefined linker amino acid. The structure parameters generated by the protein design neural network 300 for the linker positions can thus define a flexible 3D structure that enables the amino acid at the first binder position to assume an appropriate 3D spatial position relative to the target protein. Similarly the target protein defined by the conditioning data 116 can comprise the target protein followed by a string of linker amino acids.

After generating the structure parameters for the linker positions, the protein design neural network 300 can generate design data for each successive binder position until a termination criterion is satisfied. The protein design system 100 can then output the amino acid sequence and the protein structure for the binder positions as the binder protein that is predicted to bind to the target protein. The protein design system may discard the data defining the amino acids and the structure parameters for the linker positions.

Proteins generated by the protein design system 100 can be physically synthesized, e.g., using manual or automatic laboratory techniques. Such a physically synthesized protein may be used in any of a variety of possible ways. For example, a protein generated by the protein design system 100 can be used as part of a drug that is administered to a subject to achieve a therapeutic effect. As another example, a protein generated by the protein design system 100 can be used as an enzyme to catalyze an industrial process. Some further examples are listed later. Physically synthesizing a protein generated by the protein design system 100 can include experimentally validating the protein, e.g., by measuring the stability of the protein, or by measuring the real-world structure of the protein and comparing it to the structure predicted by the protein design system 100, or by evaluating the protein against the desired characteristic(s) defined by the conditioning data.

The protein design system 100 can train the protein design neural network 300 on a set of training examples. Each training example corresponds to a respective “training” protein and includes data defining the amino acid sequence of the training protein and the structure of the training protein. The structure of the training protein can be defined, e.g., by respective structure parameters for each position in the amino acid sequence of the training protein, as described above. The training examples may include “ground truth” amino acid sequences and protein structures, e.g., that have been determined by experimental techniques, e.g., protein sequencing and x-ray crystallography. When the training examples do not include conditioning data the protein designs generated by the protein design system 100 will in general be from the same distribution of the training examples, which may be selected according to the protein designs desired.

Optionally, each training example can include conditioning data that specifies characteristics of the training protein corresponding to the training example. In one example, the conditioning data can define attributes of the amino acid sequence or the structure of the training protein. In another example, the conditioning data can define a biological function of the training protein. In another example, the conditioning data can define a protein fragment that is extended by the training protein. In another example, the conditioning data can define a target protein such that the training protein binds to the target protein. In general training examples, with or without conditioning data, may be obtained from an existing protein database such as the Protein Data Bank, InterPro, Pfam; and/or training examples may be generated using a protein folding system such as a system based on AlphaFold and/or using an in-silico docking system; and/or training examples may be generated by synthesizing and characterizing proteins in a laboratory.

For each training example, the protein design system 100 can use the protein design neural network 300 to generate a respective prediction for the amino acid, the structure parameters, or both, for each position in the amino acid sequence of the training protein corresponding to the training example.

In particular, for each given position in the amino acid sequence of a training protein, the protein design neural network can process a current protein representation for the given position to generate respective probability distributions over: (i) a set of amino acids, and (ii) a set of structure parameters. The current protein representation for the position can include: (i) a representation of the amino acid and the structure parameters defined by the training example for each position that precedes the given position in the amino acid sequence, and (ii) conditioning data included in the training example.

After generating the prediction for the given position in the amino acid sequence, the protein design system 100 can determine gradients of a loss function that measures an error in the prediction. For example, the loss function can measure an error, e.g., a cross-entropy error, between: (i) the probability distribution over the set of amino acids, and (ii) the amino acid specified by the training example for the given position. As another example, the loss function can measure an error, e.g., a cross-entropy error, between: (i) the probability distribution over the set of structure parameters, and (ii) the structure parameters specified by the training example for the given position. The protein design system can determine gradients of the loss function with respect to the parameter values of the protein design neural network, e.g., using backpropagation. The protein design system can use the gradients of the loss function to adjust the current values of the protein design neural network parameters using any appropriate gradient descent optimization algorithm, e.g., Adam or RMSprop.

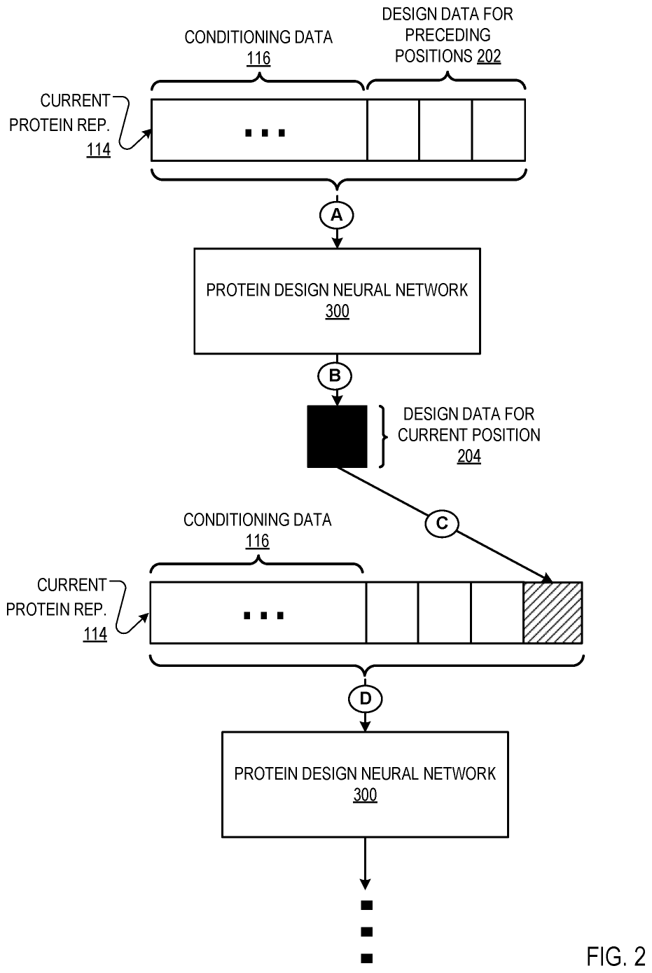

FIG. 2 illustrates examples of operations performed by the protein design system to generate design data for a current position in an amino acid sequence of a protein.

The protein design system maintains a current protein representation 114 that represents: (i) conditioning data 116, and (ii) design data for preceding positions 202 in the amino acid sequence of the protein. That is, the current protein representation 114 may comprise data defining an amino acid and structure parameters for each position in the amino acid sequence preceding the current position.

The conditioning data 116 can be any appropriate data that specifies desired characteristics of the protein being generated, e.g., characteristics of the amino acid sequence of the protein, characteristics of the structure of the protein, or characteristics of the biological function of the protein, as described above. FIG. 2 shows the conditioning data 116 notionally prepended to the design data for the amino acid sequence, but this is not a requirement and the conditioning data does not need to be associated with a particular position. For example, whilst in some implementations described later the design data may include position data, e.g. a positional embedding for each position, this is not needed for the conditioning data.

The design data for the preceding positions 202 in the amino acid sequence of the protein represents, for each preceding position that precedes the current position, the amino acid and structure parameters for the preceding position in the amino acid sequence.

The protein design system 100 provides the current protein representation 114 as an input to the protein design neural network 300 (step A), and the protein design neural network 300 processes the current protein representation 114 to generate design data 204 for the current position in the amino acid sequence (step B). The design data 204 for the current position includes: (i) an amino acid at the current position in the amino acid sequence, and (ii) structure parameters for the current position in the amino acid sequence.

The protein design system uses the design data 204 for the current position to update the current protein representation 114, e.g., by including a representation of the design data 204 for the current position in the current protein representation 114 (step C).

The protein design system 100 then provides the current protein representation 114 as an input to the protein design neural network 300 (step D) to generate design data for the next position in the amino acid sequence of the protein.

FIG. 3 shows an example architecture of a protein design neural network 300 that is included in a protein design system, e.g., the protein design system 100 described with reference to FIG. 1.

The protein design neural network 300 can autoregressively generate, for each position starting from the first position in an amino acid sequence of a protein, design data 108 defining: (i) an amino acid 100 at the position, and (ii) structure parameters 112 for the position.

To generate the design data 108 for a current position in the amino acid sequence 102 of the protein 106, the protein design neural network 300 processes a current protein representation 114. Generally, the current protein representation 114 represents at least the respective design data (i.e., amino acids and structure parameters) for any positions preceding the current position in the amino acid sequence 102. The current protein representation 114 can also include conditioning data, i.e., that specifies desired characteristics of the protein.

After the protein design neural network 300 generates the design data 108 for a current position, the protein design system 100 updates the current protein representation 114 using the design data 108 for the current position. For example, protein design system can augment the current protein representation 114 to include a representation of the design data 108 for the current position.

The protein design neural network includes an encoder neural network 400, an amino acid neural network 304, and a structure neural network 306, which are each described next.

The encoder neural network is configured to process the current protein representation 114 for a current position in the amino acid sequence, in accordance with values of a plurality of encoder neural network parameters, to generate an encoded protein representation 308. The encoded protein representation 308 defines a collection of features that characterize the current protein representation 114. The encoded protein representation 308 can be represented as an ordered collection of numerical values, e.g., a vector, matrix, or other tensor of numerical values.

An example architecture of the encoder neural network 400 is described below with reference to FIG. 4. More generally, the encoder neural network 400 can have any appropriate neural network architecture that enables it to perform its described functions, e.g., processing a current protein representation 114 for a current position in an amino acid sequence to generate an encoded protein representation 308. In particular, the encoder neural network can include any appropriate types of neural network layers (e.g., fully-connected layers, convolutional layers, attention layers, etc.) in any appropriate numbers (e.g., 5 layers, 10 layers, or 100 layers) and connected in any appropriate configuration (e.g., as a linear sequence of layers).

The amino acid neural network 304 is configured to process the encoded protein representation 308 for the current position in the amino acid sequence to generate a probability distribution over a set amino acids (proteinogenic amino acids). The set of amino acids can include, e.g., alanine, arginine, asparagine, etc. The amino acid neural network 304 selects the amino acid 100 for the current position in the amino acid sequence using the probability distribution over the set of amino acids. For example, the amino acid neural network 304 can sample the amino acid 100 for the current position in the amino acid sequence in accordance with the probability distribution over the set of amino acids.

The structure neural network 306 is configured to process the encoded protein representation 308 for the current position in the amino acid sequence, and optionally, a representation of the amino acid 100 for the current position in the amino acid sequence, to generate a probability distribution over a set of structure parameters. The structure neural network selects the structure parameters 112 for the current position in the amino acid sequence using the probability distribution over the set of possible structure parameters. For example, the structure neural network 306 can sample the structure parameters 112 for the current position in the amino acid sequence in accordance with the probability distribution over the set of structure parameters.

For example, in one implementation, the structure neural network 306 can generate a probability distribution over a set of possible backbone torsion angles, e.g., where each of the possible backbone torsion angles can be represented as a vector of three scalar values defining a phi-angle, a psi-angle, and an omega-angle. The set of possible backbone torsion angles can be a continuous space of possible torsion angles, e.g., [symbol] ³, or a discrete space, e.g., that is generated by discretizing a continuous space of possible torsion angles using any appropriate discretization scheme. The discretization scheme can be, e.g., a uniform discretization scheme, or a learned discretization scheme. A learned discretization scheme can be determined, e.g., by clustering a set of backbone torsion angles (e.g., that are measured in real-world proteins) in a continuous space of possible torsion angles (e.g., [symbol] ³) using an appropriate clustering technique (e.g., k-means clustering). After the clustering, a respective centroid of each cluster can be designated as being a vector of possible backbone torsion angles.

Optionally, the structure neural network 306 can generate the backbone torsion angles autoregressively, e.g., by generating a first backbone torsion angle (e.g., a phi-angle), then processing the first backbone torsion angle as an additional input to generate a second backbone torsion angle (e.g., a psi-angle), and then processing the first backbone torsion angle and the second backbone torsion angle as additional inputs to generate a third backbone torsion angle (e.g., an omega-angle).

The structure neural network 306 can include multiple sub-networks, where each sub-network is configured to generate a different subset of the structure parameters for the current position in the amino acid sequence. For example, the structure prediction neural network can include a first sub-network that is configured to generate a set of backbone torsion angles for the current position in the amino acid sequence. The structure prediction neural network can also include a second sub-network that is configured to process the backbone torsion angles as an additional input (e.g., in addition to the encoded protein representation 308, and optionally, the amino acid 100) to generate side chain parameters for the current position. The side chain parameters for the current position can include, e.g., side chain torsion angles, e.g., a chi1-angle, a chi2-angle, a chi3-angle, and a chi-4 angle.

The structure neural network 306 can, as described above, process an input that includes a representation of the amino acid 310 generated by amino acid neural network 304 for the current position in the amino acid sequence. Thus the structure parameters 112 generated for the current position in the amino acid sequence can be conditioned on the choice of amino acid 110 at the current position in the amino acid sequence. Conversely, in some implementations, the selection of the amino acid 110 at the current position in the amino acid sequence can be conditioned on the choice of structure parameters 112 for the current position in the amino acid sequence. For example, the amino acid neural network 304 can process an input that includes a representation of the structure parameters 112 generated by the structure neural network 306 for the current position in the amino acid sequence.

Optionally, the protein design neural network 300 can include an additional “look-ahead” neural network (not shown) that is configured to process the encoded protein representation 308 for the current position to generate an output that defines whether the current position is the last position in the amino acid sequence. For example, the look-ahead neural network can generate a probability distribution over a set of possible outputs that includes: (i) a set of possible amino acids, and (ii) an “end-of-sequence” (EOS) token. The look-ahead neural network can select an output using the probability distribution over the set of possible outputs, e.g., by sampling an output in accordance with the probability distribution. The protein design system can train the look-ahead neural network to generate: (i) the next amino acid if the current position is before the last position in the amino acid sequence, and (ii) the EOS token if the current position is the last position in the amino acid sequence.

In response to the look-ahead neural network generating an output (e.g., the EOS token) that defines the current position as the last position in the amino acid sequence of the protein, the protein design system can determine that a termination criterion is satisfied. In response to determining that a termination criterion is satisfied, the protein design system can provide the design data generated for each position up to the current position in the amino acid sequence as an output that defines the amino acid sequence and structure of the generated protein.

The amino acid neural network 304, the structure neural network 306, and the look-ahead neural network can each have any appropriate neural network architecture that enables them to perform their described functions. In particular, their neural network architectures can include any appropriate types of neural network layers (e.g., fully-connected layers, convolutional layers, attention layers, etc.) in any appropriate numbers (e.g., 5 layers, 10 layers, or 100 layers) and connected in any appropriate configuration (e.g., as a linear sequence of layers).

FIG. 4 shows an example architecture of an encoder neural network 400 that is included in a protein design neural network (e.g., the protein design neural network 300 described with reference to FIG. 3) in a protein design system (e.g., the protein design system 100 described with reference to FIG. 1).

The encoder neural network 400 is configured to process a current protein representation 114 for a current position in an amino acid sequence to generate an encoded protein representation 308 for the current position in the amino acid sequence. The protein design neural network can process the encoded protein representation 308 for the current position in the amino acid sequence, e.g., to generate data defining an amino acid and structure parameters for the current position in the amino acid sequence, as described with reference to FIG. 3.

Generally, the current protein representation 114 includes a representation of the design data (i.e., the amino acid and the structure parameters) for any positions preceding the current position in the amino acid sequence. Optionally, the current protein representation 114 can include conditioning data that specifies desired characteristics of the protein being generated by the protein design system.

The current protein representation 114 can include: (i) a sequence of data elements, and (ii) respective structure parameters (e.g., including location and rotation parameters) corresponding to some or all of the data elements in the sequence of data elements. The sequence of data elements can include one or more data elements representing the conditioning data, followed by one or more data elements representing the amino acid sequence up to the current position in the amino acid sequence.

The current protein representation 114 can represent the conditioning data by one or more data elements in a variety of ways. A few examples of representing the conditioning data by one or more data elements are described next.

In one example, the conditioning data defines an amino acid sequence and a structure of a “conditioning” protein. For example, the conditioning protein can be a protein fragment to be extended by the protein to be generated by the protein design system. As another example, the conditioning protein can be a “target” protein, where the protein to be generated by the protein design system is a “binder” protein that should bind to the target protein. The amino acid sequence of the conditioning protein can be represented by a sequence of data elements, e.g., where each data element identifies the amino acid at a corresponding position in the amino acid sequence of the conditioning protein. The structure parameters associated with each data element can represent the 3D spatial location of the corresponding amino acid in the amino acid sequence of the conditioning protein.

In another example, the conditioning data can define one or more attributes of the amino acid sequence, the structure, or the biological function of the protein to be generated by the protein design system. In this example, the current protein representation 114 can include one or more data elements representing the attributes defined by the conditioning data. For example, the current protein representation 114 can include a data element characterizing a target (desired) length of the amino acid sequence of the protein. The data element can define a particular length (e.g., 80 amino acids), or a range of lengths (e.g., 70-80 amino acids). The current protein representation 114 can include default structure parameters associated with the data elements representing attributes of the amino acid sequence, the structure, or the biological function of the protein to be generated by the protein design system can be default structure parameters. Optionally, certain data elements representing the conditioning data may not be associated with structure parameters in the current protein representation 114.

The current protein representation 114 can represent the amino acid sequence and the structure of the protein being generated by the protein design system, up to the current position in the amino acid sequence, by a sequence of data elements and corresponding structure parameters. For example, the sequence of data elements can include a designated “beginning-of-sequence” (BOS) data element, and a sequence of data elements where each data element defines an amino acid at a corresponding position in the amino acid sequence of the protein. The structure parameters associated with the BOS data element can be default structure parameters, and the structure parameters associated with a data element corresponding to an amino acid can represent the 3D spatial location of the corresponding amino acid.

The protein design system can initialize the current protein representation 114 to include: (i) a sequence of data elements and corresponding structure parameters representing the conditioning data, and (ii) the BOS data element and its corresponding default structure parameters. The protein design system can update the current protein representation when design data is generated for a current position in the amino acid sequence, e.g., by including a data element representing the amino acid at the current position and structure parameters for the current position in the current protein representation.

In some implementations, the protein design system converts the structure parameters generated by the protein design neural network for a current position in the amino acid sequence to a different format prior to including the structure parameters in the current protein representation. For example, the protein design neural network may be configured to generate structure parameters represented as backbone torsion angles, and the protein design system can convert the representation of the structure parameters to location and rotation parameters prior to including the structure parameters in the current protein representation.

The encoder neural network 400 includes an embedding layer 402 and a sequence of update blocks 404-A-N.

The embedding layer 402 of the encoder neural network 400 is configured to map each data element in the sequence of data elements of the current protein representation 114 to a corresponding embedding in an embedding space. That is, the embedding layer 402 can represent the current protein representation 114 as a collection of embeddings by replacing each data element included in the current protein representation 114 by a corresponding embedding, e.g., in accordance with a predefined mapping from data elements to embeddings (e.g., one-hot embeddings).

Optionally, for each data element in the current protein representation 114, the embedding layer 402 can combine (e.g., sum or average) the embedding of the data element with a positional embedding representing the position of the data element in the sequence of data elements of current protein representation 114.

Optionally, for each data element in the current protein representation 114, the embedding layer can process some or all of the structure parameters corresponding to the data element to generate a “structural” embedding for the data element. The embedding layer 402 can then combine (e.g., sum or average) the structural embedding corresponding to the data element with the embedding of the data element.

For example, the structure parameters for each data element in the current protein representation can include side chain parameters, in particular, side chain torsion angles, as described above. The embedding layer can generate a structural embedding for a data element by processing the side chain parameters corresponding to the data element (or data derived from the side chain parameters corresponding to the data element) using one or more neural network layers, e.g., fully-connected layers. (Data derived from the side chain parameters can include, e.g., the sine and cosine of each side chain parameter).

Each update block of the encoder neural network is configured to receive an input that includes a current embedding for each data element in the current protein representation 114, and to generate an output that includes an updated embedding for each data element in the current protein representation 114. In particular, the first update block 404-A receives the embeddings generated by the embedding layer 402, and each subsequent update block (i.e., after the first update block) receives the embeddings generated by the preceding update block in the sequence of update blocks.

Each update block, as part of updating the current embeddings to generate updated embeddings, can apply a self-attention operation over the current embeddings, e.g., a query-key-value (QKV) self-attention operation. In general a self-attention operation can be one that applies an attention mechanism to elements of an embedding to update each element of the embedding, e.g. where an input embedding is used to determine a query vector and a set of key-value vector pairs, and the updated embedding comprises a weighted sum of the values, weighted by a similarity function of the query to each respective key.

Optionally, each update block can condition the self-attention operation over the current embeddings on the structure parameters included in the current protein representation. That is, the self-attention operation can be a “geometric” self-attention operation that explicitly reasons about the 3D geometry of the amino acids in the protein, i.e., as defined by the structure parameters. More specifically, to update a given current embedding, the update block 404 determines a respective attention weight between the given current embedding and each current embedding in the collection of current embeddings, where the attention weights depend on both the current embeddings and the structure parameters. The update block 404 then updates the given current embedding using: (i) the attention weights, (ii) the collection of current embeddings, and (iii) the structure parameters.

To determine the attention weights, the update block 404 processes each current embedding to generate a corresponding “symbolic query” embedding, “symbolic key” embedding, and “symbolic value” embedding. For example, the update block 404 may generate the symbolic query embedding qᵢ^{p}, symbolic key embedding kᵢ^{p}, and symbolic value embedding vᵢ^{p} for the current embedding hᵢ corresponding to the i-th data element as:

$${q}_{i} = Linear ( {h}_{i} ) \qquad ( 1 )$$

$${k}_{i} = Linear ( {h}_{i} ) \qquad ( 2 )$$

$${v}_{i} = Linear ( {h}_{i} ) \qquad ( 3 )$$

where Linear (·) refers to linear layers having independent learned parameter values.

The update block 404 additionally processes each current embedding to generate a corresponding “geometric query” embedding, “geometric key” embedding, and “geometric value” embedding. The geometric query, geometric key, and geometric value embeddings for each current embedding are each 3-D points that are initially generated in a local reference frame, and then rotated and translated to a global reference frame using the structure parameters for the corresponding data element. For example, the update block 404 may generate the geometry query embedding qᵢ^{p}, geometric key embedding kᵢ^{p}, and geometric value embedding vᵢ^{p} for the current embedding hᵢ corresponding to the i-th data element as:

$${q}_{i}^{p} = {R}_{i} \cdot {Linear}_{p} ( {h}_{i} ) + {t}_{i} \qquad ( 4 )$$

$${k}_{i}^{p} = {R}_{i} \cdot {Linear}_{p} ( {h}_{i} ) + {t}_{i} \qquad ( 5 )$$

$${v}_{i}^{p} = {R}_{i} \cdot {Linear}_{p} ( {h}_{i} ) + {t}_{i} \qquad ( 6 )$$

where Linearₚ(·) refers to linear layers having independent learned parameter values that project hᵢ to a 3-D point (the superscript p indicates that the quantity is a 3-D point), Rᵢ denotes a rotation matrix specified by rotation parameters for the i-th data element, and tᵢ denotes location parameters for the i-th data element.

To update the current embedding hᵢ corresponding to data element i, the update block 404 may generate attention weights [aⱼ]ⱼ₌₁^{N}, where N is the total number of current embeddings and aⱼ is the attention weight between current embedding h; and current embedding hⱼ, as:

$${[ {a}_{j} ]}_{j = 1}^{N} = softmax ( {[ \frac{{q}_{i} \cdot {k}_{j}}{\sqrt{m}} + α \vert {q}_{i}^{p} - {k}_{j}^{p} {\vert}_{2}^{2} ]}_{j = 1}^{N} ) \qquad ( 7 )$$

where qᵢ denotes the symbolic query embedding for current embedding hᵢ, kⱼ denotes the symbolic key embedding for current embedding ha, m denotes the dimensionality of qᵢ and kⱼ, a denotes a learned parameter, qᵢ^{p} denotes the geometric query embedding for current embedding hᵢ, kⱼ^{p} denotes the geometry key embedding for current embedding hⱼ, and |·|₂ is an L₂ norm.

In some implementations, the update block 404 generates multiple sets of geometric query embeddings, geometric key embeddings, and geometric value embeddings, and uses each generated set of geometric embeddings in determining the attention weights.

After generating the attention weights for the current embedding hᵢ corresponding to data element i, the update block 404 uses the attention weights to update the current embedding hᵢ. In particular, the update block 404 uses the attention weights to generate a “symbolic return” embedding and a “geometric return” embedding, and then updates the current embedding using the symbolic return embedding and the geometric return embedding. The update block 404 can generate the symbolic return embedding oᵢ for current embedding hᵢ, e.g., as:

$${o}_{i} = {\sum }_{j} {a}_{j} {v}_{j} \qquad ( 8 )$$

where [aⱼ]ⱼ₌₁^{N} denote the attention weights (e.g., defined with reference to equation (7)) and each vⱼ denotes the symbolic value embedding for current embedding hⱼ. The update block 404 may generate the geometric return embedding op for current embedding hᵢ, e.g., as:

$${o}_{i}^{p} = {R}_{i}^{- 1} \cdot ( {\sum }_{j} {a}_{j} {v}_{j}^{p} - {t}_{i} ) \qquad ( 9 )$$

where the geometric return embedding oᵢ^{P} is a 3-D point, [aⱼ]ⱼ₌₁^{N} denote the attention weights (e.g., defined with reference to equation (7)), Rᵢ⁻¹ is inverse of the rotation matrix specified by the rotation parameters for data element i, and tᵢ are the location parameters for data element i. It can be appreciated that the geometric return embedding is initially generated in a global reference frame, and then rotated and translated to a local reference frame of the corresponding data element.

The update block 404 may update the current embedding hᵢ for data element i using the corresponding symbolic return embedding oᵢ (e.g., generated in accordance with equation (8)) and geometric return embedding o (e.g., generated in accordance with equation (9)), e.g., as:

$${h}_{i}^{next} = ( {h}_{i} + LayerNorm ( {h}_{i} + Linear ( {o}_{i} , {o}_{i}^{p} , \vert {o}_{i}^{p} \vert ) ) \qquad ( 10 )$$

where hᵢ^{next} is the updated current embedding for data element i, |·| is a norm, e.g., an L₂ norm, and LayerNorm(·) denotes a layer normalization operation, e.g., as described with reference to: J. L. Ba, J. R. Kiros, G. E. Hinton, “Layer Normalization,” arXiv: 1607.06450 (2016).

In some cases, certain “non-geometric” data elements in the current protein representation (e.g., that represent features of the conditioning data, e.g., a target length of the amino acid sequence) may not be associated with corresponding structure parameters. The update blocks can exclude current embeddings for non-geometric data elements from the geometric attention operations described above.

The encoder neural network 400 can generate the encoded protein representation 308 using the collection of current embeddings generated by the final update block in the sequence of update blocks. For example, the encoder neural network 400 can provide the current embedding corresponding to the last data element in the sequence of data elements of the current protein representation 114 as the encoded protein representation. As another example, the encoder neural network can generate the encoded protein representation 308 as a combination (e.g., sum or average) of the collection of current embeddings generated by the final update block.

FIG. 5 is an illustration of an unfolded protein and a folded protein. The unfolded protein is a random coil of amino acids. The unfolded protein undergoes protein folding and folds into a 3D configuration. Protein structures often include stable local folding patterns such alpha helices (e.g., as depicted by 502) and beta sheets.

FIG. 6 illustrates backbone torsion angles and side chain torsion angles in an amino acid.

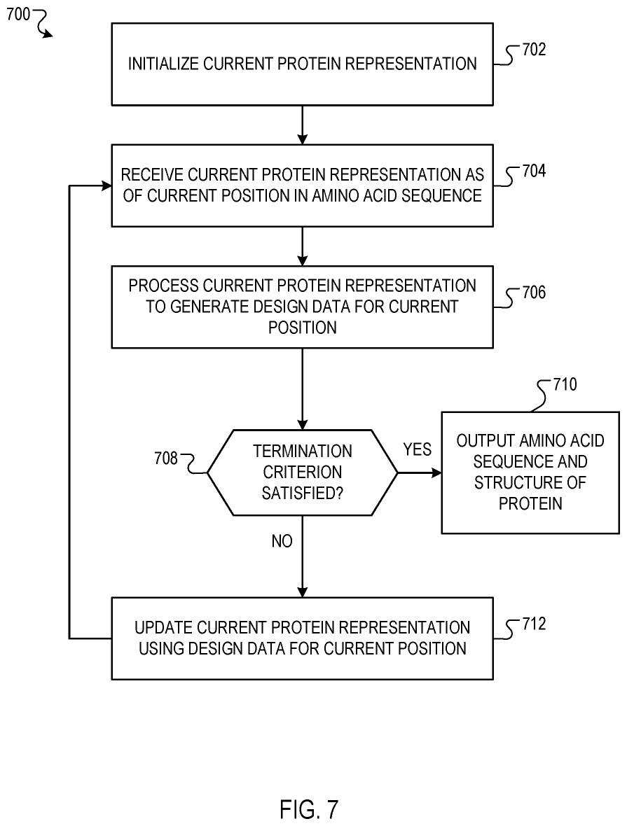

FIG. 7 is a flow diagram of an example process 700 for designing a protein by jointly generating an amino acid sequence and a structure of the protein. For convenience, the process 700 will be described as being performed by a system of one or more computers located in one or more locations. For example, a protein design system, e.g., the protein design system 100 of FIG. 1, appropriately programmed in accordance with this specification, can perform the process 700.

The system initializes a current representation of the protein (702). For example, the system can initialize the current representation of the protein to include conditioning data that specifies desired characteristics of the protein, e.g., desired attributes of the amino acid sequence of the protein, the structure of the protein, or the biological function of the protein.

The system autoregressively generates data defining the amino acid sequence and the structure of the protein using a protein design neural network. The system can perform steps 704-710, which are described next, for each position starting from the first position in the amino acid sequence of the protein.

The system receives the current representation of the protein as of, or corresponding to, the current position (704). The current representation of the protein can include a representation of respective design data for any preceding position that precedes the current position in the amino acid sequence. Design data for each preceding position can include: (i) data identifying an amino acid at the preceding position, and (ii) a set of structure parameters for the preceding position.

The system processes the current representation of the protein using the protein design neural network to generate design data for the current position (706). The design data includes: (i) data identifying an amino acid at the current position, and (ii) a set of structure parameters for the current position that characterize a three-dimensional spatial configuration of the amino acid at the current position.

The protein design neural network can include an encoder neural network, an amino acid neural network, a structure neural network, and optionally, a look-ahead neural network. The encoder neural network can process the current representation of the protein to generate an encoded representation of the protein. The amino acid neural network can process an input that includes the encoded representation of the protein to generate data identifying the amino acid at the current position. The structure neural network can process an input that includes the encoded representation to generate the structure parameters for the current position. The look-ahead neural network can process an input that includes the encoded representation to generate an output that defines whether the current position is the last position in the amino acid sequence

The system determines whether a termination criterion is satisfied (708). For example, the system can determine that a termination criterion is satisfied if the look-ahead neural network generates an output that defines the current position as the last position in the amino acid sequence.

In response to determining a termination criterion is satisfied, the system outputs the data defining the amino acid sequence and the structure of the protein (710).

In response to determining a termination criterion is not satisfied, the system updates the current representation of the protein using the design data for the current position (712). For example, the system can update the current representation of the protein to include a representation of the design data for the current position.

After updating the current representation of the protein using the design data for the current position, the system can provide the current representation of the protein for use in generating design data for a next position in the amino acid sequence of the protein (e.g., returning to step 704).

As some further examples, a protein designed by the described system may be used protein designed by the described system may be used as a drug, e.g. it may be an agonist or antagonist of a receptor or enzyme; or it may be an antibody configured to bind to an antibody target such as a virus coat protein, or a protein expressed on a cancer cell, e.g. to act as an agonist for a particular receptor or to prevent binding of another ligand and hence prevent activation of a relevant biological pathway. As another example a protein designed by the described system may be a monobody. As another example, e.g. where the conditioning data defines the amino acid sequence and the structure of a protein fragment of a CDR (complementarity-determining region) or Fc region of an antibody, a protein designed by the described system may be an antibody with the CDR or Fc region. As another example, as previously mentioned a protein designed by the system may comprise a scaffold structure supporting a protein region or domain, e.g. for binding or catalysis, defined by a protein fragment specified by the conditioning data.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method performed by one or more computers for designing a protein by jointly generating an amino acid sequence and a structure of the protein, the method comprising:
initializing a current representation of the protein;
generating data defining the amino acid sequence and the structure of the protein using a protein design neural network, comprising, for a plurality of positions in the amino acid sequence:
receiving the current representation of the protein as of the current position;
processing the current representation of the protein using the protein design neural network to generate design data for the current position that comprises: (i) data identifying an amino acid at the current position, and (ii) a set of structure parameters for the current position that characterize a three-dimensional spatial configuration of the amino acid at the current position;
updating the current representation of the protein using the design data for the current position; and
providing the current representation of the protein for use in generating design data for a next position in the amino acid sequence of the protein; and
outputting the data defining the amino acid sequence and the structure of the protein.

2. The method of claim 1, wherein for each current position after a first position in the amino acid sequence:
the current representation of the protein as of the current position comprises a representation of respective design data for each preceding position that precedes the current position in the amino acid sequence; and
the design data for each preceding position comprises: (i) data identifying an amino acid at the preceding position, and (ii) a set of structure parameters for the preceding position.

3. The method of claim 1, wherein for each current position in the amino acid sequence, the current representation of the protein as of the current position further comprises conditioning data that specifies desired characteristics of the protein.

4. The method of claim 1, wherein the protein design neural network comprises an encoder neural network, an amino acid neural network, and a structure neural network; and
wherein processing the current representation of the protein using the protein design neural network to generate design data for the current position comprises:
processing the current representation of the protein using the encoder neural network to generate an encoded representation of the protein; and
processing an input comprising the encoded representation of the protein using the amino acid neural network to generate the data identifying the amino acid at the current position; and
processing an input comprising the encoded representation of the protein using the structure neural network to generate the structure parameters for the current position.

5. The method of claim 4, wherein processing an input comprising the encoded representation of the protein using the amino acid neural network to generate data identifying the amino acid at the current position comprises:
processing the input comprising the encoded representation of the protein using the amino acid neural network to generate a probability distribution over a set of amino acids; and
selecting the amino acid at the current position using the probability distribution over the set of amino acids.

6. The method of claim 5, wherein selecting the amino acid at the current position using the probability distribution over the set of amino acids comprises:
sampling the amino acid at the current position in accordance with the probability distribution over the set of amino acids.

7. The method of claim 4, wherein processing the input comprising the encoded representation of the protein using the structure neural network to generate the structure parameters for the current position comprises:
processing the input comprising the encoded representation of the protein using the structure neural network to generate a probability distribution over a set of structure parameters; and
selecting the structure parameters for the current position using the probability distribution over the set of structure parameters.

8. The method of claim 7, wherein selecting the structure parameters for the current position using the probability distribution over the set of structure parameters comprises:
sampling the structure parameters for the current position in accordance with the probability distribution over the set of structure parameters.

9. The method of claim 7, wherein the structure neural network processes an input that comprises both: (i) the encoded representation of the protein, and (ii) data identifying the amino acid at the current position.

10. The method of claim 4, wherein processing the current representation of the protein using the encoder neural network to generate the encoded representation of the protein comprises:
processing the current representation of the protein to generate a collection of embeddings that includes a respective embedding representing an amino acid at each preceding position that precedes the current position in the amino acid sequence;
updating the collection of embeddings using one or more self-attention operations; and
after updating the collection of embeddings using the one or more self-attention operations, generating the encoded representation of the protein based on the collection of embeddings.

11. The method of claim 10, wherein the self-attention operations are conditioned on respective structure parameters for each preceding position that precedes the current position in the amino acid sequence.

12. The method of claim 1, wherein updating the current representation of the protein using the design data for the current position comprises:
updating the current representation of the protein to include a representation of the design data for the current position.

13. The method of claim 1, wherein for each of the plurality of positions in the amino acid sequence, the structure parameters for the position comprise backbone torsion angles.

14. The method of claim 1, wherein initializing the current representation of the protein comprises:
initializing the current representation of the protein to include conditioning data that specifies desired characteristics of the protein.

15. The method of claim 3, wherein the conditioning data specifies desired characteristics of the amino acid sequence of the protein, the structure of the protein, or a biological function of the protein.

16. The method of claim 14, wherein the conditioning data defines a protein fragment to be extended by the protein generated using the protein design neural network.

17. The method of claim 14, wherein the conditioning data defines a target protein that provides a binding target for the protein generated using the protein design neural network.

18. The method of claim 1, further comprising providing the protein generated using the protein neural network to be physically synthesized.

19. A system comprising:
one or more computers; and
one or more storage devices communicatively coupled to the one or more computers, wherein the one or more storage devices store instructions that, when executed by the one or more computers, cause the one or more computers to perform operations for designing a protein by jointly generating an amino acid sequence and a structure of the protein, the operations comprising:
initializing a current representation of the protein;
generating data defining the amino acid sequence and the structure of the protein using a protein design neural network, comprising, for a plurality of positions in the amino acid sequence:
receiving the current representation of the protein as of the current position;
processing the current representation of the protein using the protein design neural network to generate design data for the current position that comprises: (i) data identifying an amino acid at the current position, and (ii) a set of structure parameters for the current position that characterize a three-dimensional spatial configuration of the amino acid at the current position;
updating the current representation of the protein using the design data for the current position; and
providing the current representation of the protein for use in generating design data for a next position in the amino acid sequence of the protein; and
outputting the data defining the amino acid sequence and the structure of the protein.

20. One or more non-transitory computer storage media storing instructions that when executed by one or more computers cause the one or more computers to perform operations for designing a protein by jointly generating an amino acid sequence and a structure of the protein, the operations comprising:
initializing a current representation of the protein;
generating data defining the amino acid sequence and the structure of the protein using a protein design neural network, comprising, for a plurality of positions in the amino acid sequence:
receiving the current representation of the protein as of the current position;
processing the current representation of the protein using the protein design neural network to generate design data for the current position that comprises: (i) data identifying an amino acid at the current position, and (ii) a set of structure parameters for the current position that characterize a three-dimensional spatial configuration of the amino acid at the current position;
updating the current representation of the protein using the design data for the current position; and
providing the current representation of the protein for use in generating design data for a next position in the amino acid sequence of the protein; and
outputting the data defining the amino acid sequence and the structure of the protein.

21. (canceled)
